(19) United States
(12) Patent Application Publication
(10) Pub. No.: US 2002/0031533 A1
(43) Pub. Date: Mar. 14, 2002

(54) Method of using fibers as an antipollution agent, in particular in a cosmetic composition

(75) Inventor: Isabelle Afriat, New York, NY (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/861,599

(22) Filed: May 22, 2001

(30) Foreign Application Priority Data
May 22, 2000 (FR) 0006510

(51) Int. Cl. A61K 7/00

(52) U.S. Cl. 424/401

(57) ABSTRACT

Cosmetic composition comprising fibers for topical application to the skin, the scalp and/or the hair protect keratinous materials against the harmful effects of pollution. The fibers have a length (L) ranging from 1 μm to 10 mm and a cross section within a circle having a diameter (D) ranging from 1 nm to 100 μm. In particular, the fibers may be polyamide fibers.

### BACKGROUND OF THE INVENTION

1. Field of the Invention

The present application relates to the use of fibers, in a cosmetic composition, as antipollution agent or for the preparation of a composition intended for protecting keratinous materials against the harmful effects of pollution.

2. Discussion of the Background

Some urban surroundings are regularly subjected to peaks of pollution. Individuals in their daily environment, and particularly in urban areas, may be subjected to multiple attacks on the keratinous materials of their bodies, and in particular the skin, the scalp and the hair, by various aerial pollutants.

Among the pollutants which may exert deleterious effects on keratinous fibers, toxic gases such as ozone, carbon monoxide, oxides of nitrogen or oxides of sulphur are one of the major constituents. It has been observed that these toxic gases promote desquamation of the keratinous fibers, making them dirty and dull. Likewise, these gases cause cellular asphyxia at the level of these keratinous materials.

It is known, moreover, that heavy metals (lead, cadmium, mercury) are atmospheric pollutants whose emissions have notably increased, in particular in urban or industrial surroundings. In addition to certain toxic effects which are specific to them, heavy metals have the property of reducing the activity of the means of cellular defense against free radicals (see for example R. S. Dwivedi, J. Toxicol. Cut. & Ocular Toxical. 6(3), 183-191 (1987)). Thus, heavy metals aggravate the toxic effects of gaseous pollutants by reducing the efficacy of the natural means of defense, and cause acceleration of the phenomenon of cellular ageing. This is true in particular for the keratinous materials and in particular the skin, the scalp and the hair which are in direct and permanent contact with the external surroundings.

Thus, the harmful effects of pollution on the keratinous materials affect cellular respiration and lead to accelerated ageing of the skin, with a dull complexion and the premature formation of wrinkles or fine lines, and also to a reduction in the vitality of the hair which also take on a dull appearance. In addition, because of the pollution, the skin and the hair become dirty more quickly. Furthermore, the pollution may cause skin allergy phenomena.

Thus, the need exists for compositions which make it possible to avoid the harmful effects due to pollutants (gases or heavy metals), so as to protect the keratinous materials.

### SUMMARY OF THE INVENTION

It has now been observed, quite surprisingly, that the use of fibers, in particular polyamide fibers, made it possible to protect keratinous materials such as the skin, scalp and hair from the effects of pollutants.

Indeed, cosmetic compositions containing short polyamide fibers, those giving the said compositions a velvety feel and a good cosmetic behaviour, are known for example from the document JP07-196440. However, no document describes that the fibers can have properties of protecting against pollution.

Thus, the present invention is a method of protecting keratinous materials such as the skin, scalp and hair from the effects of pollutants using cosmetic composition for topical application to the skin, the scalp and/or the hair, containing fibers as an antipollution agent.

The expression “topical application” is understood here to mean an external application to the keratinous materials, which are in particular the skin, the scalp, the eyelashes, the eyebrows, the nails and the mucous membranes.

The subject of the invention is also the use of fibers for the preparation of a composition intended to protect the keratinous materials against the harmful effects of pollution. In addition to a good antipollution effect, the compositions containing the fibers have good cosmetic properties: comfort on application, texture easy to spread and smooth.

The fibers which can be used in the composition of the invention may be hydrophilic or hydrophobic fibers of synthetic or natural, and inorganic or organic origin. These fibers may be short or long, individual or organized, for example plaited. They can have any shape or morphology, and in particular can have a circular or polygonal (square, hexagonal or octagonal) cross section, according to the specific application envisaged. In particular, their ends are blunt and/or smooth in order to prevent injury.

In particular, the fibers may have a length (L) ranging from 1 μm (0.001 mm) to 10 mm, preferably from 0.1 μm to 5 mm and better still from 0.1 mm to 1.5 mm. Their cross section may be within a circle having a diameter (D) ranging from 1 nm (0.001 μm) to 100 μm, preferably ranging from 1 nm (0.001 μm) to 50 μm and better still from 5 μm to 40 μm. Preferably, the fibers used according to the present invention have an aspect ratio, that is to say an L/D (length/diameter) ratio ranging from 3.5 to 2500, better still from 5 to 500 or even better from 5 to 150.

The yarn count of the fibers is often given in denier or decitex. Denier is the weight in grams for 9 km of thread. Preferably, the fibers used according to the invention have a yarn count ranging from 0.15 to 30 denier, and better still from 0.18 to 18 denier. The aspect ratio, the yarn count and the morphology of the fibers are the three factors which are important for defining a fiber.

The fibers may be those used in the manufacture of textiles, and in particular silk fibers, cotton fibers, wool fibers, flax fibers, cellulose fibers which are extracted in particular from wood, vegetables or algae, polyamide fibers (Nylon ®), modified cellulose fibers (rayon, viscose, acetate, in particular rayon acetate), poly(p-phenylene terephthamide) fibers, in particular Kevlar® fibers, acrylic fibers, in particular poly(methyl methacrylate) or poly(2-hydroxyethyl methacrylate) fibers, polyolefin fibers, and in particular polyethylene or polypropylene fibers, glass fibers, silica fibers, aramid fibers, carbon fibers, in particular in graphite form, Teflon® fibers, insoluble collagen fibers, polyester fibers, poly(vinyl chloride) or poly(vinylidene chloride) fibers, poly(vinyl alcohol) fibers, polyacrylonitrile fibers, chitosan fibers, polyurethane fibers or poly(ethylene phthalate) fibers, fibers formed from a mixture of polymers such as those mentioned above, such as polyamide/polyester fibers.

It is also possible to use the resorbable synthetic fibers used in surgery, such as the fibers prepared from glycolic acid and caprolactone (Monocryl from the company Johnson & Johnson); resorbable synthetic fibers of the copolymer of lactic acid and of glycolic acid type (Vicryl from the company Johnson & Johnson); terephthalic polyester fibers (Ethibond from the company Johnson & Johnson) and stainless steel wires (Acier from the company Johnson & Johnson). It is also possible to use mixtures of the abovementioned fibers.

Moreover, the fibers may or may not be surface-treated and may or may not be coated. As coated fibers which can be used in the invention, there may be mentioned polyamide fibers coated with copper sulphide for an antistatic effect (for example R-STAT from the company Rhodia) or another polymer allowing a particular organization of the fibers (specific surface treatment) or a surface treatment which induces colour/hologram effects (Lurex fiber from the company Sildorex for example).

Depending on their properties, the fibers used according to the present invention may be introduced into an aqueous medium, an oily medium or into a powder.

The fibers which can be used according to the invention are preferably chosen from polyamide fibers, poly(p-phenyleneterephthamide) fibers, cotton fibers and mixtures thereof. Their length may range from 0.1 to 10 mm, preferably from 0.1 to 1 mm, their mean diameter may range from 5 to 50 μm and the aspect ratio preferably ranges from 5 to 150.

In particular, it is possible to use the polyamide fibers marketed by Etablissements P. Bonte under the name Polyamide 0.9 Dtex 0.3 mm, having a mean diameter of 15 to 20 μm, a yam count of about 0.9 dtex (0.81 denier) and a length ranging from 0.3 mm to 1.5 mm. It is also possible to use poly(p-phenylene terephthamide) fibers having a mean diameter of 12 μm and a length of about 1.5 mm such as those sold under the name Kevlar Floc by the company Du Pont Fibers. These polyamide fibers are preferably introduced into an oily medium or, using the dry route, into a powder.

It is also possible to use cotton fibers having a mean diameter of 20 μm, a length ranging from 0.3 mm and an aspect ratio of 15.

The fibers may be present in the composition according to the invention in a quantity ranging, for example, from 0.1 to 20% by weight, preferably from 0.5 to 15% by weight and better still from 2 to 15% by weight relative to the total weight of the composition.

The composition may also contain other antipollution active agents such as, for example, the sphingolipids described in the document EP-A-0 577 718.

The compositions for topical application, and in particular the cosmetic compositions, used according to the invention contain a physiologically acceptable medium, that is to say a medium compatible with the skin, the lips, the scalp, the eyelashes, the eyes, the nails and/or the hair. This physiologically acceptable medium may consist more particularly of water and optionally a physiologically acceptable organic solvent chosen, for example, from lower alcohols comprising from 1 to 4 carbon atoms such as ethanol, isopropanol, propanol, butanol; polyethylene glycols having from 6 to 80 ethylene oxides; polyols such as propylene glycol, isoprene glycol, butylene glycol, glycerin, sorbitol. This may also be an anhydrous medium, in particular an oily medium containing oils and/or fatty substances other than oils.

The physiologically acceptable medium has a pH compatible with the skin, preferably ranging from 3 to 8 and better still from 4.5 to 7.

When the composition comprises an aqueous or aqueous-alcoholic medium, it is possible to add a fatty (or oily) phase to this medium so that the compositions of the invention are smoother and more nourishing.

Thus, the compositions according to the invention containing antipollution agents as defined above may be provided in all the galenic forms conventionally used for topical application and in particular in the form of aqueous, aqueous-alcoholic or oily solutions, of oil-in-water (O/W) or water-in-oil (W/O) or multiple emulsions, of aqueous or oily gels, of liquid, pasty or solid anhydrous products, or of dispersions of a fatty phase in an aqueous phase with the aid of spherules, it being possible for these spherules to be polymeric nanoparticles such as nanospheres and nanocapsules, or lipid vesicles of the ionic and/or nonionic type. These compositions are prepared according to customary methods.

In addition, the compositions used according to the invention may be fluid to a greater or lesser degree and may have the appearance of a white or coloured cream, an ointment, a milk, a lotion, a serum, a paste, a foam. They may be optionally applied to the skin in aerosol form. They may also be provided in solid form, and for example in the form of a stick.

As oils which can be used in the compositions of the invention, there may be mentioned inorganic oils such as liquid paraffin; oils of plant origin such as the liquid fraction of shea butter, sunflower oil; oils of animal origin such as perhydrosqualene; synthetic oils such as hydrogenated polyisobutene; nonvolatile or volatile silicone oils such as cyclomethicones like cyclopentasiloxane; and fluorinated oils such as perfluoropolyethers. It is also possible to use, as fatty substances other than oils, fatty alcohols, fatty acids, waxes. The oily phase of the emulsion may also contain gums such as silicone gums, resins and in particular silicone resins, silicone elastomers such as the products marketed under the name “KSG” by the company Shin-Etsu, under the name “Trefil” by the company Dow Coming or under the name “Gransil” by the company General Electric.

According to a specific embodiment of the invention, the composition containing the fibers is a water-in-oil (W/O) or oil-in-water (O/W) emulsion. The proportion of the oily phase of the emulsion may range from 5 to 80% by weight, and preferably from 5 to 50% by weight relative to the total weight of the composition. The oils, the emulsifiers and the co-emulsifiers used in the composition in the form of an emulsion are chosen from those conventionally used in the cosmetic or dermatological field. The emulsifier and the co-emulsifier are generally present in the composition in a proportion ranging from 0.3 to 30% by weight, and preferably from 0.5 to 20% by weight relative to the total weight of the composition. The emulsion may, in addition, contain lipid vesicles.

The emulsions generally contain at least one emulsifier chosen from amphoteric, anionic, cationic or nonionic emulsifiers which are used alone or in the form of a mixture. The emulsifiers are appropriately chosen according to the emulsion to be obtained (W/O or O/W).

For the W/O emulsions, there may be mentioned, for example, as emulsifiers, dimethicone copolyols such as the mixture of cyclomethicone and dimethicone copolyol, sold under the name “DC 5225 C” by the company Dow Corning, and the alkyldimethicone copolyols such as laurylmethicone copolyol sold under the name “Dow Corning 5200 Formulation Aid” by the company Dow Coming and cetyldimethicone copolyol sold under the name Abil EM 90® by the company Goldschmidt. It is also possible to use, as emulsifier for W/O emulsions, a crosslinked elastomeric solid organopolysiloxane comprising at least one oxyalkylenated group, such as those obtained according to the procedure of Examples 3, 4 and 8 of the document U.S. Pat. No. 5,412,004 and of the examples of the document U.S. Pat. No. 5,811,487, in particular the product of Example 3 (example of synthesis) of U.S. Pat. No. 5,412,004, and such as that marketed under the reference KSG 21 by the company Shin-Etsu.

In a known manner, the cosmetic or dermatological composition of the invention may also contain customary adjuvants in the cosmetic or dermatological field, such as hydrophilic or lipophilic gelling agents, hydrophilic or lipophilic active agents, preservatives, antioxidants, solvents, perfumes, fillers, screening agents, bactericides, odour absorbers, colouring matter or salts. The quantities of these various adjuvants are those conventionally used in the field considered, and are for example from 0.01 to 20% of the total weight of the composition. These adjuvants, depending on their nature, may be introduced into the fatty phase, into the aqueous phase andlor into the lipid spherules.

As active agents, it is possible to add to the fibers used in the composition according to the invention other agents known to promote pollution control. As other antipollution agents, there may be mentioned for example the metallothioneins described in the document EP-A-557 042, the sphingolipids described in the document EP-A-577 718, and any other compound having the property of avoiding the harmful effects of pollutants.

As fillers which may be used in the composition of the invention, there may be mentioned for example, in addition to the pigments, silica powder; talc; polyamide particles and in particular those sold under the name ORGASOL by the company Atochem; polyethylene powders; microspheres based on acrylic copolymers, such as those made of ethylene glycol dimethacrylate/lauryl methacrylate copolymer which are sold by the company Dow Corning under the name POLYTRAP; expanded powders such as hollow microspheres and in particular the microspheres marketed under the name EXPANCEL by the company Kemanord Plast or under the name MICROPEARL F 80 ED by the company Matsumoto; powders of natural organic materials such as crosslinked or noncrosslinked maize, wheat or rice starches, such as the powders of starch crosslinked with octenylsuccinic anhydride, which are marketed under the name DRY-FLO by the company National Starch; microbeads of silicone resin such as those marketed under the name TOSPEARL by the company Toshiba Silicone; and mixtures thereof. These fillers may be present in quantities ranging from 0 to 20% by weight 10 and preferably from 1 to 10% by weight relative to the total weight of the composition.

The compositions used according to the invention may in particular constitute a healthcare and/or make-up product. They may be used in particular for protecting the body, in particular keratinous materials, against the effects of pollution, in particular for improving cellular respiration and/or for reducing desquamation and/or for avoiding making the keratinous materials, and in particular the skin, dull or dirty.

Thus, another subject of the invention consists in a method of cosmetic treatment for protecting keratinous materials against the effects of pollution, consisting in applying to the keratinous materials an effective quantity of a cosmetic composition containing fibers.

The subject of the invention is also a method for the cosmetic treatment of keratinous materials for improving their cellular respiration and/or for reducing their desquamation and/or for avoiding making them dull and/or making them dirty, consisting in applying to the keratinous materials an effective quantity of a cosmetic composition containing fibers.

The following examples serve to illustrate the invention without, however, having a limiting character. The names are, depending on the case, chemical names or CTFA names (International Cosmetic Ingredient Dictionary and Handbook) and the quantities are in percent by weight unless otherwise stated.

### EXAMPLE 1

W/O Emulsion

| | |
|---|---|
| Phase A: | |
| microcrystalline wax | 1.2% |
| hydrogenated polyisobutene | 4.6% |
| propylparaben (preservative) | 0.01% |
| polyaminopropyl biguanide (preservative) | 1% |
| magnesium sulphate | 0.7% |
| silica | 0.5% |
| polymethylsesquioxane | 0.4% |
| ethylene/acrylic acid copolymer | 0.6% |
| acrylates copolymer | 0.04% |
| Phase B: | |
| cyclomethicone/disteardimonium | 2% |
| hectorite/alcohol (85/10/5 mixture sold | |
| under the name Bentone Gel VS-5V by | |
| Elementis Specialties) | |
| polyamide fibers | 10% |
| (Polyamide 0.9 Dtex, 0.3 mm - company Paul | |
| Bonte) | |
| cyclopentasiloxane | 9% |
| dimethicone/vinyl dimethicone | 2% |
| crosspolymer/dimethicone (KSG 16 sold by | |
| Shin-Etsu) | |
| Phase C: | |
| dimethicone/vinyl dimethicone | 2% |
| crosspolymer/dimethicone (KSG 21 sold by | |
| Shin-Etsu) | |
| cyclopentasiloxane | 9% |
| Phase D: | |
| glycerin | 5% |
| water | qs 100% |

Procedure:

Phase A and phase B are prepared separately, they are mixed and the mixture is passed through a triple roll mill several times. The mixture obtained is added to phase C and the whole is homogenized, with stirring. The emulsion is then prepared, with stirring, by pouring, in small portions, the aqueous phase D into the mixture of phases A, B and C.

### EXAMPLE 2

O/W Emulsion

| Phase A: | |
|---|---|
| 1/premix A′ | |
| microcrystalline wax | 1.2% |
| hydrogenated polyisobutene | 4.6% |
| silica | 0.5% |
| ethylene/acrylic acid copolymer | 0.6% |
| acrylates copolymer | 0.04% |
| polymethylsesquioxane | 0.4% |
| dimethicone/vinyl dimethicone cross-polymer | 2.57% |
| and dimethicone (KSG 6 from the | |
| company Shin-Etsu) | |
| preservatives | 0.015% |
| 2/premix A″ | |
| polyamide fibers | 10% |
| (Polyamide 0.9 Dtex, 0.3 mm - company Paul | |
| Bonte) | |
| cyclomethicone/disteardimonium | 2% |
| hectorite/alcohol (85/10/5 mixture sold | |
| under the name Bentone Gel VS-5V by | |
| Elementis Specialties) | |
| cyclopentasiloxane | 28% |
| dimethicone/vinyl dimethicone | 2% |
| crosspolymer and dimethicone (KSG 16 sold | |
| by Shin-Etsu) | |
| Phase B: | |
| preservatives | 1% |
| carbomer | 0.5% |
| acrylates/C10-30 alkyl acrylate | 0.5% |
| crosspolymer (Pemulen from the company | |
| Goodrich) | |
| water | qs 100% |
| Phase C: | |
| triethanolamine | 0.6% |
| water | 2% |

Procedure:

Premixes A′ and A″ of phase A are first of all mixed by incorporating premix A″ into premix A′ and by passing the mixture through a triple roll mill several times. Phase B is prepared in parallel by dispersing the Pemulen and the carbomer in water supplemented with preservatives, with vigorous stirring (moritz type). The emulsion is prepared by pouring, in small portions, phase A into phase B, with stirring. The mixture is then neutralized by adding phase C.

### EXAMPLE 3

O/W Emulsion

| Phase A: | |
|---|---|
| polyamide fibers | 12% |
| (Polyamide 0.9 Dtex, 0.3 mm - company Paul | |
| Bonte) | |
| cyclomethicone/disteardimonium | 2% |
| hectorite/alcohol (85/10/5 mixture sold | |
| under the name Bentone Gel VS-5V by | |
| Elementis Specialties) | |
| cyclopentasiloxane | 12% |
| dimethicone/vinyl dimethicone | 4% |
| crosspolymer and dimethicone (KSG 16 sold | |
| by Shin-Etsu) | |
| Phase B: | |
| preservatives | 1% |
| carbomer | 0.3% |
| acrylates/C10-30 alkyl acrylate | 0.3% |
| crosspolymer (Pemulen from the company | |
| Goodrich) | |
| water | qs 100% |
| Phase C: | |
| triethanolamine | 0.6% |
| water | 2% |

Procedure:

The constituents of phase A are mixed and this mixture is passed through a triple roll mill several times. Phase B is prepared in parallel by dispersing, with vigorous stirring (moritz type), the Pemulen and the carbomer in water supplemented with preservatives. The emulsion is prepared by pouring, in small portions, phase A into phase B, with stirring. The mixture is then neutralized by adding phase C.

Test for Demonstrating the Protective Effect of the Fibers

Antipollution Efficacy on Reconstructed Skin

The emulsions of Examples 1 and 2 (2 mg/cm²) were applied to the surface of the epidermis of reconstructed skin and left in contact with them for 30 minutes at room temperature. The epidermis of reconstructed skin which are used are sold by the company EPISKIN (LYON, France) and the culture media are those included in the kit sold by the supplier. The epidermis were used at the D 13 Differentiation stage.

After the 30 minutes of contact, particles radio-labelled with carbon 14 were deposited on the epidermis and left in contact with them for 2 hours in the usual maintenance medium for epidermis. The epidermis were then removed from their maintenance medium and washed several times with PBS buffer (=phosphate buffered saline). The washes made it possible to free the epidermes of particles which are weakly adsorbed without removing the emulsion initially applied. The levels of residual radiolabelled particles were then evaluated by measuring the radioactivity of the carbon 14 added to the particles. The table below gives the results, in %, of residual particles relative to the quantity of particles deposited:

| | TABLE |
|---|---|
| | % relative to the quantity |
| | of particles deposited |
| Area not treated | 36.2% |
| Area treated with emulsion | 14.3% |
| of Example 1 | |
| Area treated with emulsion | 26.9% |
| of Example 2 | |

These results show that the emulsions according to the invention allow good protection of the skin against the polluting particles and that the water-in-oil emulsion of Example 1 still has a better protection than the oil-in-water emulsion of Example 2.

The priority document of the present application, French patent application 00 06510, filed May 22, 2000, is incorporated herein by reference. What is claimed as new and is intended to be secured by Letters Patent is:

1. A method of protecting keratinous materials from aerial pollutants comprising:
treating said keratinous materials with a composition comprising fibers in an amount effective to protect said keratinous materials.

2. The method of claim 1, wherein said keratinous materials are selected from the group consisting of skin, scalp, and hair.

3. The method of claim 1, wherein said aerial pollutants are selected from the group consisting of ozone, carbon monoxide, oxides of nitrogen, oxides of sulfur, heavy metals and mixtures thereof.

4. The method of claim 1, wherein said fibers are selected from the group consisting of silk fibers, cotton fibers, wool fibers, flax fibers, cellulose fibers, polyamide fibers, modified cellulose fibers, poly(p-phenylene terephthamide) fibers, acrylic fibers, polyolefin fibers, glass fibers, silica fibers, aramid fibers, carbon fibers, Teflon® fibers, insoluble collagen fibers, polyester fibers, poly(vinyl chloride) fibers, poly(vinylidene chloride) fibers, poly(vinyl alcohol) fibers, polyacrylonitrile fibers, chitosan fibers, polyurethane fibers, poly(ethylene phthalate) fibers, fibers formed from a mixture of polymers, resorbable synthetic fibers, and mixtures thereof.

5. The method of claim 1, wherein the fibers are selected from the group consisting of polyamide fibers, poly-(p-phenylene terephthalamide) fibers, cotton fibers, and mixtures thereof.

6. The method of claim 1, wherein the fibers have a length of from 1 μm to 10 mm.

7. The method of claim 6, wherein the fibers have a length of from 0.1 mm to 1.5 mm.

8. The method of claim 1, wherein the fibers have a cross sectional diameter of from 1 nm to 100 μm.

9. The method of claim 8, wherein the fibers have a cross sectional diameter of from 5 μm to 40 μm.

10. The method of claim 1, wherein the fibers have an aspect ratio of from 3.5 to 2500.

11. The method of claim 10, wherein the fibers have an aspect ratio of from 5 to 150.

12. The method of claim 1, wherein the fibers have a yarn count of from 0.15 to 30 denier.

13. The method of claim 12, wherein the fibers have a yarn count of from 0.18 to 18 denier.

14. The method of claim 1, wherein the fibers are coated and/or surface-treated.

15. The method of claim 1, wherein the fibers are present in an amount of 0.1 to 20% by weight relative to the total weight of the composition.

16. The method of claim 1, wherein the composition further comprises a physiologically acceptable medium.

17. The method of claim 16, wherein the composition is in the form of an aqueous solution, an aqueous-alcoholic solution, an oily solution, a oil-in-water emulsion, a water-in-oil emulsion, a multiple emulsion, an aqueous gel, an oily gel, a pasty anhydrous product, a solid anhydrous product, a dispersion of a fatty phase in an aqueous phase in the presence of spherules.

18. The method of claim 1, wherein the composition is in the form of an emulsion.

19. The method of claim 1, wherein when said keratinous material is skin or scalp, said protecting is improving the cellular respiration of the keratinous material and/or reducing the desquamation of the keratinous material.

20. The method of claim 1, wherein said protecting prevents the keratinous material from becoming dull and/or dirty.

21. The method of claim 1, wherein said treating comprises applying an effective amount of the composition to the keratinous material.

22. The method of claim 1, wherein the composition is a healthcare or cosmetic composition.

23. The method of claim 1, wherein the composition has the form of a white or colored cream, an ointment, a milk, a lotion, a serum, a paste, a foam, a solid, and a foam.

24. The method of claim 1, wherein the composition further comprises another antipollution agent.
